(19) United States
(12) Patent Application Publication
(10) Pub. No.: US 2021/0059892 A1
(43) Pub. Date: Mar. 4, 2021

(54) PILLOW TYPE NECK MASSAGE DEVICE WITH ADJUSTABLE MASSAGE INTENSITY

(71) Applicant: DMBH Co., Ltd., Bucheon-si (KR)

(72) Inventor: YONG GIL PARK, Seongnam-si (KR)

(73) Assignee: DMBH Co., Ltd., Bucheon-si (KR)

(21) Appl. No.: 16/587,040

(22) Filed: Sep. 29, 2019

(30) Foreign Application Priority Data
Aug. 30, 2019 (KR) 10-2019-0107160

(51) Int. Cl. A61H 7/00

(52) U.S. Cl. CPC A61H 7/004; A61H 7/007; A61H 2201/1215; A61H 2015/0014; A61H 2201/0134; A61H 2201/5061; A61H 2205/04

(57) ABSTRACT

A pillow type neck massage device with adjustable massage intensity, includes: a base board; an upper cover coupled to the base board to support a user's rear neck region; and a massage unit disposed on the base board to massage the user's rear neck region, wherein the massage unit has a body shape measurement module adapted to measure a shape of the user's rear neck region at the time when the user rests his or her head on the pillow type neck massage device.

### CROSS REFERENCE TO RELATED APPLICATION OF THE INVENTION

The present application claims the benefit of Korean Patent Application No.10-2019-0107160 filed in the Korean Intellectual Property Office on Aug. 30, 2019, the entire contents of which are incorporated herein by reference.

### FIELD OF THE INVENTION

The present invention relates to a pillow type neck massage device with adjustable massage intensity, and more particularly, to a pillow type neck massage device with adjustable massage intensity that is capable of recognizing a shape of a user's rear neck portion corresponding to a massage region to allow massage intensity to be appropriately adjustable according to the recognized shape of the user's rear neck portion.

### BACKGROUND OF THE RELATED ART

Generally, a pillow is one of bedding which supports a user's head while the user is resting or sleeping, and in conventional practices, most of pillows are configured to fill their body with soft materials, thereby providing a given cushion force for the user.

However, the conventional pillows just serve to support the user's head, without any separate functions, so that they fail to satisfy consumers who want to have their pillow providing various functions.

Also, many people suffer their physical pain, especially, neck and shoulder stiffness, after work.

Such physical pain is resulted from over work, stress, or bad posture for long hours. In this case, the cervical spine becomes abnormally bent, and also, shoulder muscles become severely stiff.

At this time, it is good to use a pillow for correction of the cervical spine while the user is sleeping, and before sleeping, otherwise, the stiff muscles have to be relaxed through massage.

Recently, functional massage pillows having a neck massage function have been developed to relax a user's neck muscles.

However, most of the functional massage pillows are pillow type massage devices that serve to massage a user's neck and occipital bone region, irrespective of his or her body shape or biological state, according to time and massage intensity set in advance by him or her or on the pillows.

The conventional functional massage pillows are operated with set driving patterns and massage intensity, while not considering the user's body shape (that is, neck thickness, shape, etc.), thereby having limitations in providing efficient massage.

### SUMMARY OF THE INVENTION

Accordingly, the present invention has been made in view of the above-mentioned problems occurring in the prior art, and it is an object of the present invention to provide a pillow type neck massage device with adjustable massage intensity that is capable of measuring a shape of a user's rear neck portion corresponding to a massage region to allow massage intensity to be automatically provided according to the measured shape of the user's rear neck portion.

To accomplish the above-mentioned object, according to the present invention, there is provided a pillow type neck massage device with an adjustable massage intensity, including: a base board; an upper cover coupled to the base board to support a user's rear neck region; and a massage unit disposed on the base board to massage the user's rear neck region, wherein the massage unit has a body shape measurement module adapted to measure a shape of the user's rear neck region at the time when the user rests his or her head on the pillow type neck massage device.

According to the present invention, desirably, the massage unit includes: two massage rods for massaging given portions of the user's rear neck region; a vertical driving assembly for moving the two massage rods in a vertical direction (in a direction of z axis) toward the rear neck at the time when the user rests his or her head on the pillow type neck massage device; a left and right driving assembly for moving the two massage rods in a left and right direction (in a direction of x axis) with respect to the user at the time when he or she rests his or her head on the pillow type neck massage device; and an up and down driving assembly for moving the two massage rods in an up and down direction (in a direction of y axis) with respect to the user at the time when he or she rests his or her head on the pillow type neck massage device, and the two massage rods are coupled to force sensors for measuring forces or loads applied thereto during massage.

According to the present invention, desirably, the massage unit further includes: a massage intensity measurer for measuring massage intensity of the two massage rods in real time through the force sensors; and a controller for receiving the measured massage intensity from the massage intensity measurer to control driving of the vertical driving assembly, the left and right driving assembly and the up and down driving assembly, so that while massaging, the massage intensity is adjustable.

According to the present invention, desirably, the massage unit is operated, while adjusting the massage intensity according to the user's shape measured through the body shape measurement module.

According to the present invention, desirably, the body shape measurement module is adapted to measure the user's neck shape from coordinate values of the z axis at the time when a pressure value reaches a given value, while the two massage rods are moving in the direction of z axis at one or more set measurement positions for measuring the user's neck shape.

According to the present invention, desirably, the body shape measurement module includes: a transmission part for outputting a transmission signal to the controller to move the two massage rods to one or more set measurement positions sequentially; a measurement part for measuring moving displacement from the given value at the time when the pressure values measured from the force sensors of the two massage rods reach the given value at the respective measurement positions for measuring the user's neck shape; a storage part connected to the measurement part to store the coordinate values measured on the measurement positions through the measurement part; and a module controller for setting appropriate massage intensity with the neck shape measured from the measurement part to provide the set massage intensity to the controller.

According to the present invention, desirably, the force sensors are constituted of strain gauge load cells.

According to the present invention, desirably, the load cells recognize the moving displacement of z axis to provide information on the ratio of forces to displacement if the forces are applied.

According to the present invention, desirably, when the two massage rods are moved in the up and down direction (in the direction of y axis) through the up and down driving assembly, the left and right driving assembly and the vertical driving assembly are mounted on the up and down driving assembly, so that the left and right driving assembly and the vertical driving assembly are moved together with the up and down driving assembly.

According to the present invention, desirably, when the two massage rods are moved in the vertical direction (in the direction of z axis) through the vertical driving assembly, the left and right driving assembly is mounted on the vertical driving assembly, so that the left and right driving assembly is moved together with the vertical driving assembly.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a perspective view showing a pillow type neck massage device with adjustable massage intensity according to the present invention;

FIG. 2 is a schematic block diagram showing a massage unit of the pillow type neck massage device according to the present invention;

FIG. 3 is a block diagram showing a body shape measurement module of the pillow type neck massage device according to the present invention;

FIG. 4 is a block diagram showing a control procedure of the pillow type neck massage device according to the present invention;

FIG. 5 illustrates sectional views (a) and (b) showing measurements of a surface property determination part according to the present invention;

FIG. 6 is an exploded perspective view showing a base board and an upper cover of the pillow type neck massage device according to the present invention;

FIG. 7 is a plan view showing the base board of the pillow type neck massage device according to the present invention;

FIG. 8 is a perspective view showing an up and down driving assembly of the pillow type neck massage device according to the present invention;

FIG. 9 is a front view showing a vertical driving assembly of the pillow type neck massage device according to the present invention;

FIG. 10 is a perspective view showing the vertical driving assembly of the pillow type neck massage device according to the present invention;

FIG. 11 is a front view showing a left and right driving assembly of the pillow type neck massage device according to the present invention; and

FIG. 12 is a perspective view showing the left and right driving assembly of the pillow type neck massage device according to the present invention.

### DETAILED DESCRIPTION OF THE PREFERRED EMBODIMENTS

Now, the present invention will be given with reference to the attached drawings. In the description, the thicknesses of the lines or the sizes of the components shown in the drawing may be magnified for the clarity and convenience of the description.

Further, the terms as will be discussed later are defined in accordance with the functions of the present invention, but may be varied under the intention or regulation of a user or operator. Therefore, they should be defined on the basis of the whole scope of the present invention.

Before the present invention is disclosed and described, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure.

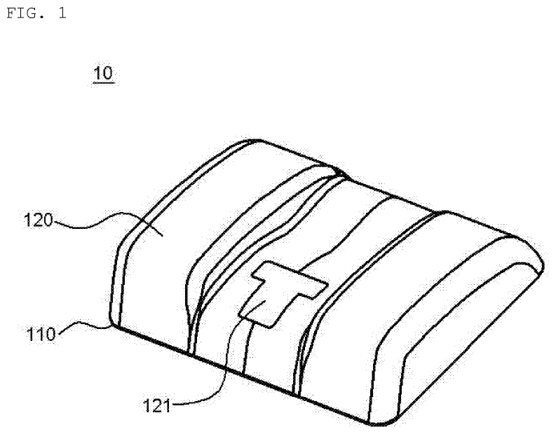

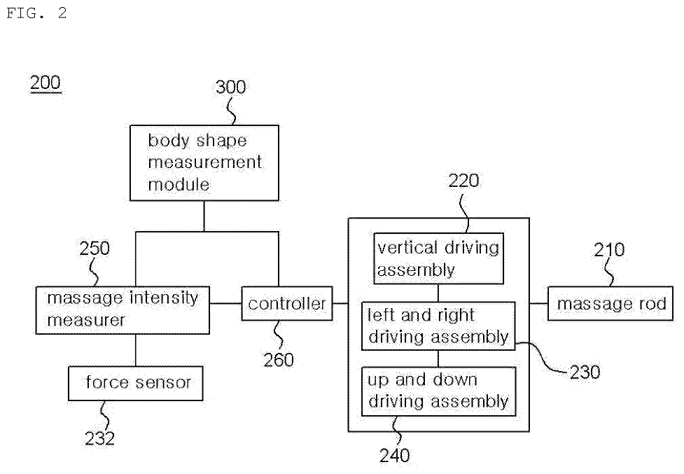

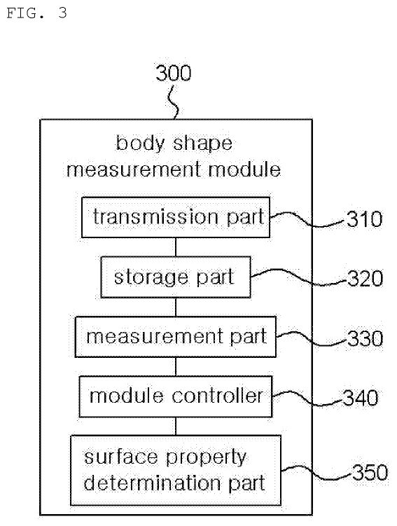

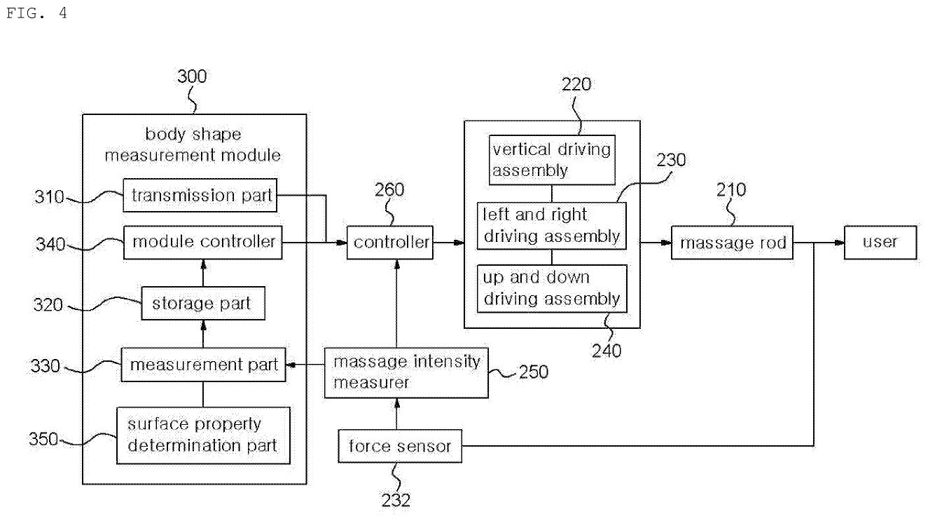

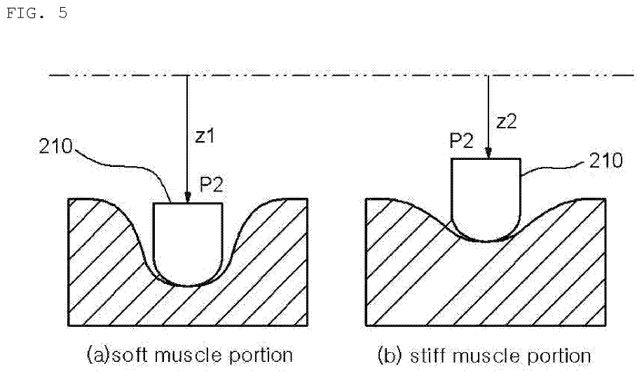

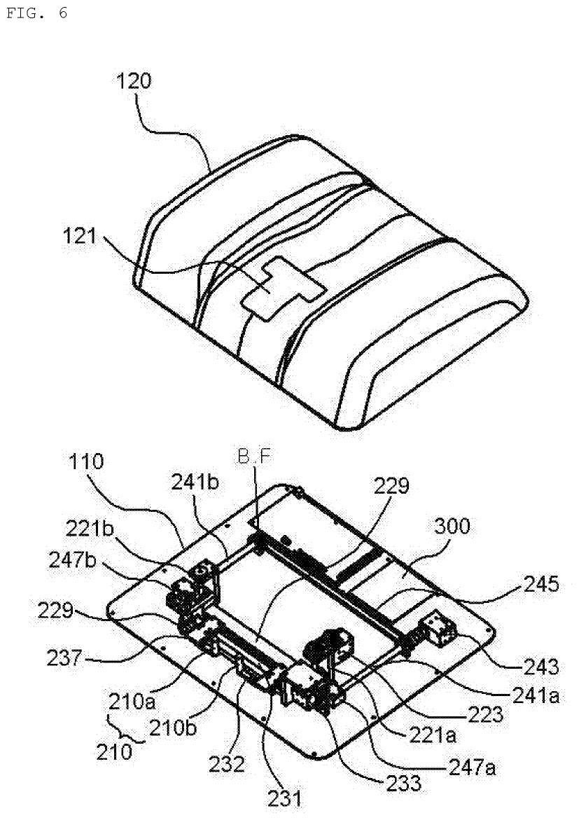

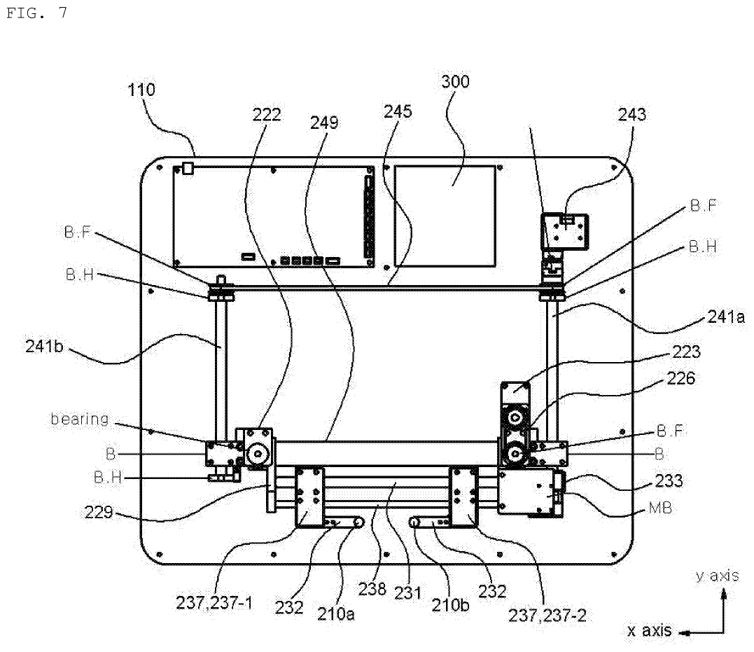

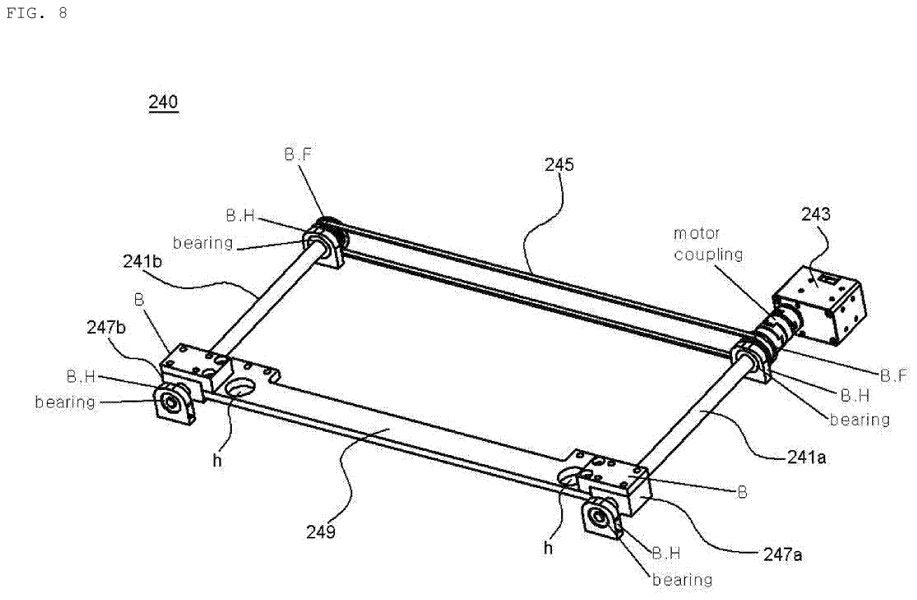

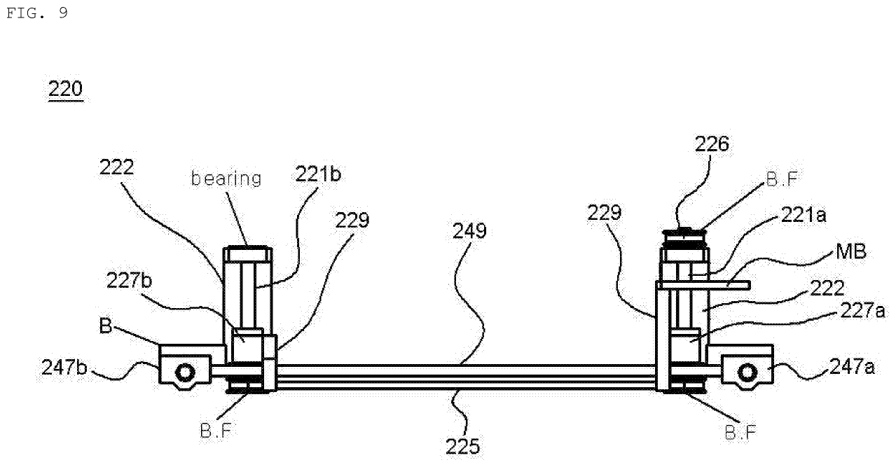

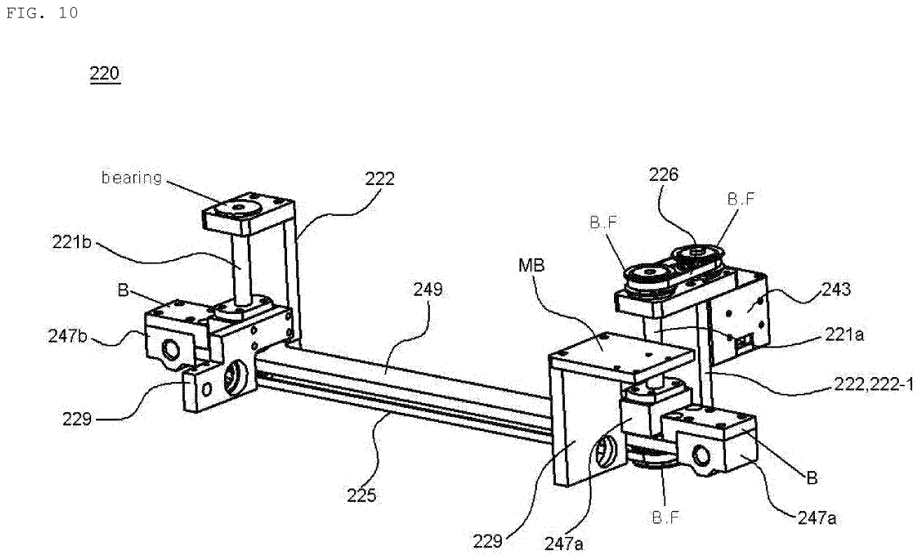

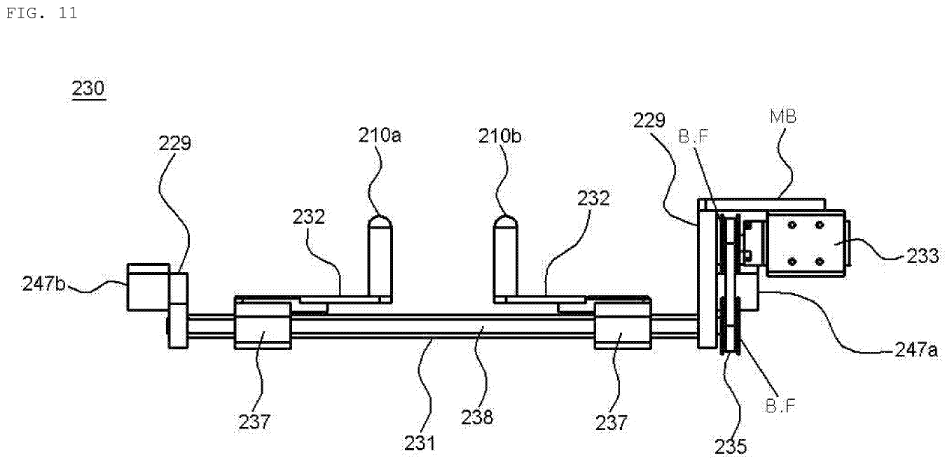

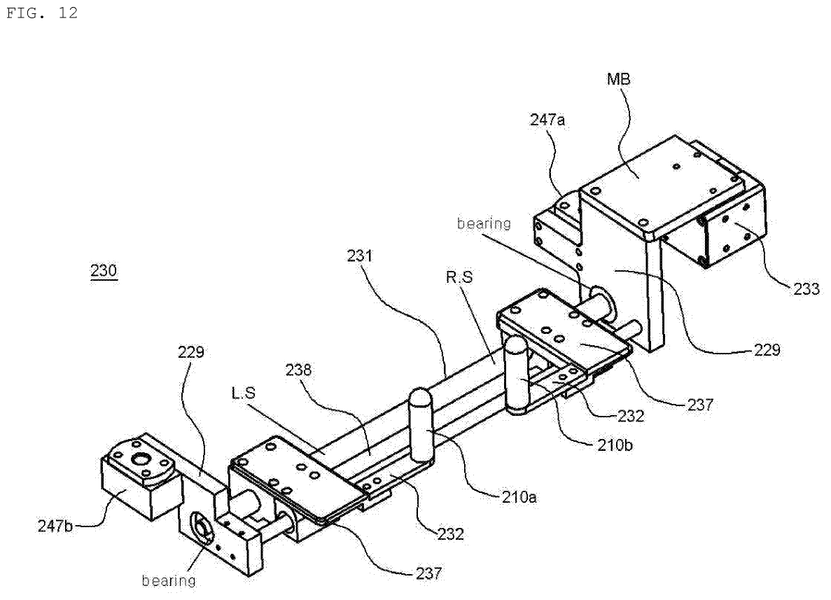

FIG. 1 is a perspective view showing a pillow type neck massage device with adjustable massage intensity according to the present invention, FIG. 2 is a schematic block diagram showing a massage unit of the pillow type neck massage device according to the present invention, FIG. 3 is a block diagram showing a body shape measurement module of the pillow type neck massage device according to the present invention, FIG. 4 is a block diagram showing a control procedure of the pillow type neck massage device according to the present invention, FIG. 5 illustrates sectional views (a) and (b) showing measurements of a surface property determination part according to the present invention, FIG. 6 is an exploded perspective view showing a base board and an upper cover of the pillow type neck massage device according to the present invention, FIG. 7 is a plan view showing the base board of the pillow type neck massage device according to the present invention, FIG. 8 is a perspective view showing an up and down driving assembly of the pillow type neck massage device according to the present invention, FIG. 9 is a front view showing a vertical driving assembly of the pillow type neck massage device according to the present invention, FIG. 10 is a perspective view showing the vertical driving assembly of the pillow type neck massage device according to the present invention, FIG. 11 is a front view showing a left and right driving assembly of the pillow type neck massage device according to the present invention, and FIG. 12 is a perspective view showing the left and right driving assembly of the pillow type neck massage device according to the present invention.

As shown in FIG. 1, the present invention relates to a pillow type neck massage device that is adapted to massage muscles around a user's neck in a state where he or she rests his or her head thereon, thereby achieving good blood circulation, gentle metabolism, and stress relief.

According to the present invention, the pillow type neck message device 10 includes a base board 110 and an upper cover 120 coupled to the base board 110 to support a user's rear neck region.

At this time, an outer cloth of the upper cover 120 is made of latex or memory foam.

Further, a massage unit 200 is disposed on the base board 110 to massage the user's rear neck region.

The massage unit 200 has a body shape measurement module 300 adapted to measure a shape of the user's rear neck region at the time when he or she rests his or her head on the pillow type neck massage device 10.

At this time, the massage unit 200 includes two massage rods 210 for massaging given portions of the rear neck, a vertical driving assembly 220 for moving the two massage rods 210 in a vertical direction (in a direction of z axis) toward the rear neck at the time when the user rests his or her head on the pillow type neck massage device 10, a left and right driving assembly 230 for moving the two massage rods 210 in a left and right direction (in a direction of x axis) with respect to the user at the time when he or she rests his or her head on the pillow type neck massage device 10, and an up and down driving assembly 240 for moving the two massage rods 210 in an up and down direction (in a direction of y axis) with respect to the user at the time when he or she rests his or her head on the pillow type neck massage device 10.

Further, the two massage rods 210 are a left massage rod 210a for massaging a given portion on the left side of the rear neck and a right massage rod 210b for massaging a given portion on the right side of the rear neck.

Furthermore, force sensors 232 are provided to measure forces or loads applied to the two massage rods 210, so that it is possible to adjust massage intensity while massaging.

Also, the massage unit 200 includes a massage intensity measurer 250 for measuring the massage intensity of the two massage rods 210 in real time through the force sensors 232.

Further, the massage unit 200 includes a controller 260 for receiving the measured massage intensity from the massage intensity measurer 250 to control driving of the vertical driving assembly 220, the left and right driving assembly 230 and the up and down driving assembly 240, so that while massaging, the massage intensity can be adjusted.

Moreover, the two massage rods 210 have plate springs coupled to ends of x axis moving bodies 237 of the left and right driving assembly 230 as will be discussed later.

Accordingly, the massage unit 200 is operated, while adjusting the massage intensity according to the user's shape measured through the body shape measurement module 300.

Further, the body shape measurement module 300 is adapted to measure the user's neck shape from coordinate values of the z axis at the time when a pressure value reaches a given value P1, while the two massage rods 210 are moving in the direction of z axis at one or more set measurement positions for measuring the user's neck shape.

Also, the body shape measurement module 300 includes a transmission part 310 for outputting a transmission signal to the controller 260 to move the two massage rods 210 to one or more set measurement positions sequentially, a measurement part 330 for measuring moving displacement from the given value P1 at the time when the pressure values measured from the force sensors 232 of the two massage rods 210 reach the given value P1 at the respective measurement positions for measuring the user's neck shape, a storage part 320 connected to the measurement part 330 to store the coordinate values measured on the measurement positions through the measurement part 300, and a module controller 340 for setting appropriate massage intensity P2 with the neck shape measured from the measurement part 330 to provide the set massage intensity P2 to the controller 260.

Further, the body shape measurement module 300 includes a surface property determination part 350 for comparing, while the measurement part 330 is measuring moving displacement from the massage intensity P2 at the measurement positions during the massage with the massage intensity P2, the measured data with the data of the storage part 320 to determine the surface properties of the user's rear neck.

Also, the surface property determination part 350 is adapted to compare displacement values for the massage intensity P2 at the respective measurement positions with the moving displacement of the given value P1 and to thus determine the surface properties of the user's rear neck according to the compared displacement values. In detail, the displacement values are divided into three groups, and in case of the group having the biggest displacement value, it is determined that muscles are knotted. Further, in case of the group having the intermediate displacement value, it is determined that muscles are tense, and in case of the group having the smallest displacement value, it is determined that muscles are relieved.

In this case, the displacement values are difference values between the coordinate values of z axis at the massage intensity P2 and the coordinate values of z axis at the given value P1.

Moreover, the surface property determination part 350 is adapted to transmit the user's surface property data to the controller 260, and the controller 260 increases the number of massage times and massage intensity at the position where the muscles are knotted on the basis of the user's surface property data.

If it is assumed that the number of massage times is 1 at the position where the muscles are relieved, the number of massage times is 2 at the position where the muscles are tense, and the number of massage times is 3 at the position where the muscles are knotted.

Moreover, if it is assumed that massage intensity is 1 at the position where the muscles are relieved, massage intensity is 2 at the position where the muscles are tense, and massage intensity is 3 at the position where the muscles are knotted.

On the other hand, the force sensors 232 are constituted of strain gauge load cells.

At this time, the load cells recognize the moving displacement of z axis to provide information on the ratio of forces to displacement if the forces are applied.

When the two massage rods 210 are moved in the up and down direction (in a direction of y axis) through the up and down driving assembly 240, the left and right driving assembly 230 and the vertical driving assembly 220 are mounted on the up and down driving assembly 240, so that the left and right driving assembly 230 and the vertical driving assembly 220 are moved together with the up and down driving assembly 240.

When the two massage rods 210 are moved in the vertical direction (in a direction of z axis) through the vertical driving assembly 220, the left and right driving assembly 230 and the vertical driving assembly 220 are mounted on the vertical driving assembly 220, so that the left and right driving assembly 230 is moved together with the vertical driving assembly 220.

Accordingly, the two massage rods 210 are vertically moved, while moving in the up and down or left and right direction, to massage the user's rear neck and thus to relieve his or her stress.

Also, the upper cover 120 has an exposure portion 121 from which the two massage rods 210 are exposed.

Further, the exposure portion 121 is covered with a soft cover to prevent the massage unit 200 from being exposed to the outside and to keep the user's neck from coming into direct contact with the two massage rods 210, thereby removing inconveniences in using the pillow type neck massage device 10.

On the other hand, the up and down driving assembly 240 includes first and second up and down rotary shafts 241a and 241b rotatably spaced apart from each other by a given distance in parallel with the up and down direction (the direction of y axis) on both sides of the massage unit 200.

At this time, housings B.H are provided to support both ends of the respective first and second up and down rotary shafts 241a and 241b, and the housings B.H are provided with bearings.

Accordingly, the bearings fitted to the housings B.H are coupled to the first and second up and down rotary shafts 241a and 241b.

Further, an up and down driving motor 243 is disposed on top of the first up and down rotary shaft 241a to rotate the first up and down rotary shaft 241a.

A first belt 245 is adapted to connect the first and second up and down rotary shafts 241a and 241b with each other to rotate the second up and down rotary shaft 241b.

At this time, belt pulleys B.F are fitted to the first and second up and down rotary shafts 241a and 241b to prevent the first belt 245 from escaping from the first and second up and down rotary shafts 241a and 241b.

In detail, the belt pulleys B.F are fitted to the first and second up and down rotary shafts 241a and 241b, and further, the first belt 245 is mounted on the belt pulleys B.F, so that even during the motor is driven, the first belt 245 does not escape from the first and second up and down rotary shafts 241a and 241b.

Also, first and second up and down moving bodies 247a and 247b are fitted to the first and second up and down rotary shafts 241a and 241b and are moved simultaneously to the up and down direction (in the direction of y axis) through the operation of the up and down driving motor 243.

Further, a connection plate 249 is disposed between the first and second up and down moving bodies 247a and 247b by means of brackets B in such a manner as to mount the vertical driving assembly 220 thereon.

On the other hand, the vertical driving assembly 220 includes first and second vertical rotary shafts 221a and 221b passing through both sides of the connection plate 249 in such a manner as to be rotatable in parallel with the vertical direction (the direction of z axis).

At this time, through holes h are formed on the connection plate 249, and bearings are fitted to the through holes h, so that they fit the first and second vertical rotary shafts 221a and 221b thereto.

Further, a vertical driving motor 223 is provided to rotate the first vertical rotary shaft 221a.

Furthermore, a second belt 225 is adapted to connect the first and second vertical rotary shafts 221a and 221b with each other to rotate the second vertical rotary shaft 221b.

At this time, the second belt 225 is disposed under the connection plate 249.

Further, belt pulleys B.F on which the second belt 225 is mounted are fitted to the first and second vertical rotary shafts 221a and 221b to prevent the second belt 225 from escaping from the first and second vertical rotary shafts 221a and 221b even during the motor is driven.

Also, first and second vertical moving bodies 227a and 227b are fitted to the first and second vertical rotary shafts 221a and 221b and are moved simultaneously to the vertical direction (to the direction of z axis) through the operation of the vertical driving motor 223.

Further, connection brackets 229 are coupled to the first and second vertical moving bodies 227a and 227b in such a manner as to mount the left and right driving assembly 230 thereon.

Also, the vertical driving assembly 220 includes support brackets 222 coupled to the connection plate 249 to support front surfaces of the first and second vertical rotary shafts 221a and 221b.

At this time, bearings are fitted to the support brackets 222 and are thus coupled to top end peripheries of the first and second vertical rotary shafts 221a and 221b.

The vertical driving motor 223 is coupled to the support bracket 222-1 supporting the first vertical rotary shaft 221a.

Further, a third belt 226 is adapted to connect a motor shaft of the vertical driving motor 223 and the first vertical rotary shaft 221a to rotate the first vertical rotary shaft 221a.

At this time, belt pulleys B.F are fitted to the first vertical rotary shaft 221a on the upper (front) side of the support bracket 222-1 and to the motor shaft of the vertical driving motor 223.

In detail, the third belt 226 is mounted on the belt pulleys B.F fitted to the first vertical rotary shaft 221a and to the vertical driving motor 223, so that it does not escape from the first vertical rotary shaft 221a and the vertical driving motor 223 even during the motor is driven.

On the other hand, the left and right driving assembly 230 includes a left and right rotary shaft 231 to which both ends of the connection bracket 229 are rotatably coupled.

Also, a left and right driving motor 233 is provided to rotate the left and right rotary shaft 231.

A left screw L.S and a right screw R.S are formed on the left and right portions around the center of the left and right rotary shaft 231.

Accordingly, the x axis moving bodies 237 are disposed on both sides of the left and right rotary shaft 231, so that a distance between them becomes long or short through the operation of the left and right driving motor 233.

Further, a motor bracket MB protrudes in a right direction from the connection bracket 229 coupled to the first vertical moving body 227a and is thus coupled to the left and right driving motor 233.

Also, a fourth belt 235 is adapted to connect a motor shaft of the left and right driving motor 233 and the left and right rotary shaft 231 to rotate the left and right rotary shaft 231.

At this time, belt pulleys B.F on which the fourth belt 235 is disposed are fitted to the left and right rotary shaft 231 and the motor shaft of the left and right driving motor 233, thereby preventing the fourth belt 235 from escaping from the left and right rotary shaft 231 and the left and right driving motor 233 even during the motor is driven.

According to the present invention, the pillow type neck massage device 10 is configured to couple the connection plate 249 to the first and second up and down moving bodies 247a and 247b by means of brackets B.

Further, the first and second vertical rotary shafts 221a and 221b to which the first and second vertical moving bodies 227a and 227b are fitted are coupled to the connection plate 249.

Furthermore, the connection brackets 229 are coupled to the first and second vertical moving bodies 227a and 227b, and the left and right rotary shaft 231 to which the x axis moving bodies 237 are fitted are coupled to the connection brackets 229.

When the left and right driving assembly 230 and the vertical driving assembly 220 are mounted on the up and down driving assembly 240 to allow the two massage rods 210 to be moved in the up and down directions through the up and down driving assembly 240, in detail, they are moved, together with the up and down driving assembly 240.

When the left and right driving assembly 230 is mounted on the vertical driving assembly 220 to allow the two massage rods 210 to be moved in the vertical direction through the vertical driving assembly 220, also, it is moved, together with the vertical driving assembly 220.

While the two massage rods 210 are being moved in the up and down direction through the up and down driving assembly 240, accordingly, they are moved in the vertical direction toward the user's neck through the vertical driving assembly 220, and the distance between the two massage rods 210 is increased or decreased through the left and right driving assembly 230, so that the muscles around the user's neck region can be massaged.

As described above, the pillow type neck massage device with adjustable massage intensity according to the present invention is configured to measure the shape of the user's neck region to be massaged, to automatically provide massage intensity according to the measured shape of the user's neck region, to automatically sense the massage intensity, and to adjust the massage intensity really applied to the user, thereby providing more comfortable and effective massage for the user.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A pillow type neck massage device with adjustable massage intensity, comprising:
a base board (110);
an upper cover (120) coupled to the base board (110) to support a user's rear neck region; and
a massage unit (200) disposed on the base board (110) to massage the user's rear neck region,
wherein the massage unit (200) has a body shape measurement module (300) adapted to measure a shape of the user's rear neck region at the time when the user rests his or her head on the pillow type neck massage device (10).

2. The pillow type neck massage device according to claim 1, wherein the massage unit (200) comprises:
two massage rods (210) for massaging given portions of the user's rear neck region;
a vertical driving assembly (220) for moving the two massage rods (210) in a vertical direction (in a direction of z axis) toward the rear neck at the time when the user rests his or her head on the pillow type neck massage device (10);
a left and right driving assembly (230) for moving the two massage rods (210) in a left and right direction (in a direction of x axis) with respect to the user at the time when he or she rests his or her head on the pillow type neck massage device (10) ; and
an up and down driving assembly (240) for moving the two massage rods (210) in an up and down direction (in a direction of y axis) with respect to the user at the time when he or she rests his or her head on the pillow type neck massage device (10), and
the two massage rods (210) are coupled to force sensors (232) for measuring forces or loads applied thereto during massage.

3. The pillow type neck massage device according to claim 2, wherein the massage unit (200) further comprises:
a massage intensity measurer (250) for measuring massage intensity of the two massage rods (210) in real time through the force sensors (232); and
a controller (260) for receiving the measured massage intensity from the massage intensity measurer (250) to control driving of the vertical driving assembly (220), the left and right driving assembly (230) and the up and down driving assembly (240), so that while massaging, the massage intensity is adjustable.

4. The pillow type neck massage device according to claim 3, wherein the massage unit (200) is operated, while adjusting the massage intensity according to the user's shape measured through the body shape measurement module (300).

5. The pillow type neck massage device according to claim 4, wherein the body shape measurement module (300) is adapted to measure the user's neck shape from coordinate values of the z axis at the time when a pressure value reaches a given value (P1), while the two massage rods (210) are moving in the direction of z axis at one or more set measurement positions for measuring the user's neck shape.

6. The pillow type neck massage device according to claim 5, wherein the body shape measurement module (300) comprises:
a transmission part (310) for outputting a transmission signal to the controller (260) to move the two massage rods (210) to one or more set measurement positions sequentially;
a measurement part (330) for measuring moving displacement from the given value (P1) at the time when the pressure values measured from the force sensors (232) of the two massage rods (210) reach the given value (P1) at the respective measurement positions for measuring the user's neck shape;
a storage part (320) connected to the measurement part (330) to store the coordinate values measured on the measurement positions through the measurement part (300); and
a module controller (340) for setting appropriate massage intensity (P2) with the neck shape measured from the measurement part (330) to provide the set massage intensity (P2) to the controller (260).

7. The pillow type neck massage device according to claim 4, wherein the force sensors (232) are constituted of strain gauge load cells.

8. The pillow type neck massage device according to claim 7, wherein the load cells recognize the moving displacement of z axis to provide information on the ratio of forces to displacement if the forces are applied.

9. The pillow type neck massage device according to claim 8, wherein when the two massage rods (210) are moved in the up and down direction (in the direction of y axis) through the up and down driving assembly (240), the left and right driving assembly (230) and the vertical driving assembly (220) are mounted on the up and down driving assembly (240), so that the left and right driving assembly (230) and the vertical driving assembly (220) are moved together with the up and down driving assembly (240).

10. The pillow type neck massage device according to claim 9, wherein when the two massage rods (210) are moved in the vertical direction (in the direction of z axis) through the vertical driving assembly (220), the left and right driving assembly (230) is mounted on the vertical driving assembly (220), so that the left and right driving assembly (230) is moved together with the vertical driving assembly (220).
